(19) United States
(12) Patent Application Publication
(10) Pub. No.: US 2005/0283020 A1
(43) Pub. Date: Dec. 22, 2005

(54) Process for the preparation of 2-aryl propionic acids

(76) Inventors: Raghunath Vitthal Chaudhari, Pune (IN); Seayad A., Pune (IN); Jayasree Seayad, Pune (IN)

Correspondence Address: GAUTHIER & CONNORS, LLP, 225 FRANKLIN STREET, BOSTON, MA, 02110, US

(21) Appl. No.: 11/149,804

(22) Filed: Jun. 10, 2005

Related U.S. Application Data
(63) Continuation of application No. 09/628,158, filed on Jul. 28, 2000, now abandoned.

(51) Int. Cl. C07C 51/16

(52) U.S. Cl. 562/410

(57) ABSTRACT

The present invention provides for the preparation of 2-aryl propionic acids, which comprises the steps of: reacting an aryl compound selected from an arylalkyl halide having general formula I, aryl alcohol having general formula II or aryl substituted olefins having general formula III, as shown in the accompanying drawings, wherein, R₁ is aryl, substituted aryl, naphthyl or substituted naphthyl groups, R₂, R₃, R₄ and R₅ are independently hydrogen, alkyl, aryl, arylalkyl or cycloaliphatic groups with or without substituents and X is other a halogen atom selected from chlorine, bromine, iodine with a halide promoter, an organic acid, water and a palladium catalyst in an organic solvent selected from ketones or cyclic ethers in carbon monoxide atmosphere under homogeneous conditions, at a temperature ranging between 30 to 130° C., for a period ranging between 0.3 to 4 hrs, at pressures ranging between 50 to 1500 psig, cooling the reaction mixture to ambient temperature, flushing the reaction vessel with inert gas, removing the solvent by conventional methods, and separating the catalyst and isolating 2 aryl propionic acid having formula IV as shown in the accompanying drawings, wherein, R₁ is aryl, substituted aryl, naphthyl or substituted naphthyl groups, R₂, R₃, R₄ and R₅ are independently hydrogen, alkyl, aryl, arylalkyl, cycloaliphatic groups with or without substituents.

### FIELD OF THE INVENTION

This invention relates to an unproved process for the preparation of 2-aryl propionic acids. Particularly, this invention relates to an improved process for conversion of aryl alkyl halides having general formula I, aryl alcohols of general formula II or aryl substituted olefins of general formula III wherein, R₁ may be aryl, substituted aryl, naphthyl or substituted naphthyl, R₂, R₃, R₄ and R₅ may independently be hydrogen, alkyl, aryl, arylalkyl, cycloaliphatic with or without substitutents, and X may be halogen atom such as chlorine, bromine, iodine, to their corresponding 2-aryl propionic acids having general formula IV using a homogeneous palladium catalyst system.

### PRIOR ART

A majority of the 2-aryl propionic acids are well-known non-steroidal anti-inflammatory drugs; Ibuprofen and Naproxen being two important examples. The conventional synthesis of ibuprofen involves six steps which use hazardous chemicals like cyanides and the waste materials produced requires lot of down stream treatments for disposal. Recently, Hoechst Celanese Corporation has developed a novel environmentally benign three step catalytic process for the synthesis of ibuprofen, in which carbonylation of para isobutylphenyl ethanol (p-IBPE) is the key step. In the process described in patented literature (EP 0,400,892A3, EP 0,284,310A1), the catalysts used were mainly Pd(PPh₃)₂Cl₂ or PdCl₂ or Pd (OAc)₂ along with excess phosphine ligands in a biphasic system consisting of 10% aqueous HCl as the promoter and p-IBPE dissolved in a solvent as the organic phase. The main drawback of this process is the low reaction rates (TOF=25-35 h⁻¹) and low selectivity to ibuprofen (56-69%) under mild conditions (130° C., 1000 psig). Higher selectivity (>95%) was obtained only at very high pressures of 2000 to 4500 psig of carbon monoxide and the rates still remained low.

U.S. Pat. No. 5,536,874 and the publication J. Chem. Tech. Biotechnol, 1997, 70, 83-91, describes the carbonylation of p-IBPE in a two-phase system wherein one phase is an aqueous medium which contains a water soluble palladium complex and an acid promoter. These processes also have disadvantages such as low reaction rates (TOF=0.1 to 0.4 h⁻¹) and low ibuprofen selectivity (59-74%) under mild reaction conditions (90° C., 450 to 900 psig). The patents EP 0 338 852 and U.S. Pat. No. 5,055,611, describes preparation of 2-arylpropionic acids by the carbonylation of aryl alkyl halides using PdCl₂(PPh₃)₂ as the catalyst precursor along with 5% aqueous HCl as the promoter. In these cases also, only low reaction rate and low ibuprofen selectivity were achieved. Another pathway for the preparation of 2-aryl propionic acids which is more rewarding is the carbonylation of aryl olefins which can be easily obtained from the catalytic cracking of corresponding saturated hydrocarbons and is more economical. Ali and Alper reported in a publication J. Mol. Catal. 1992, 77, 7-13, the carbonylation of aryl olefins using Pd(OAc)₂/dppb/PPh₃/HCOOH catalyst system. But the reaction rate (TOF=2.2 h⁻¹) and 2-aryl propionic acid selectivity (15-20%) were too low, the major product being the 3-aryl propionic acid. More recently, the U.S. Pat. No. 5,260,477 disclosed a process for the carbonylation of p-isobutyl styrene to ibuprofen using PdCl₂(PPh₃)₂/10% HCl, under very high CO pressures (300 bar at 120° C.) which again gave low reaction rate (TOF≅25 h⁻¹) and ibuprofen selectivity (89%). Another U.S. Pat. No. 5,315,026 reported the carbonylation of p-isobutyl styrene to ibuprofen using a PdCl₂/CuCl₂/(+)-neomenthyl diphenylphosphine/10% HCl catalyst system which gave good ibuprofen selectivity (<98%), but very low reaction rate (TOF≅25 h⁻¹) under 30-200 psig CO pressure at 100° C. The publications New. J. Chem. 1997, 21. 529-531 and Catal. Lett., 1997, 47, 43-46 revealed the carbonylation of aryl olefins to 2-aryl propionic acids using a biphasic catalyst system (PdCl₂/TPPTS) under 50 bar CO pressure at 65-100° C. which also gave low reaction rates (25-50 h⁻¹) and low selectivity (60-75%).

The inventors of the present invention have observed that the use of a new homogeneous catalyst system comprising a palladium compound, an organic acid and a halide promoter provides an improved catalyst system for the carbonylation of arylalkyl halides of general formula I, aryl alcohols of general formula II, or aryl substituted olefins of the general formula III to the corresponding 2-arylpropionic acids. The use of such a catalyst system gives high reaction rates and high selectivity to 2-arylpropionic acids under mild reaction conditions.

### OBJECTS OF THE INVENTION

The main object of the invention is to provide an improved process for the preparation of 2-aryl propionic acids by the carbonylation of arylalkyl halides, aryl alcohols or aryl substituted olefins.

Another object of the invention is to provide a process wherein novel catalyst system under mild reaction conditions in a homogeneous medium are involved. Still another object of the invention is to provide an improved process wherein high reaction rates and high productivity of 2-aryl propionic acid are achieved. Yet another object of the invention relates to an improved process which provides very high selectivity of 2-aryl propionic acid even under lower pressures of carbon monooxide.

### SUMMARY

To meet the above objectives, the invention provides a process for the preparation of 2-aryl propionic acids, said method comprising the steps of treating aryl alkyl halides or aryl alcohols or aryl substituted olefins with a halide promoter, organic acid, water and palladium catalyst in a organic solvent and cooling the reaction mixture to obtain aryl propionic acid.

### BRIEF DESCRIPTION OF THE ACCOMPANYING DRAWINGS

FIG. 1 represents formula I of aryl alkyl halides.

FIG. 2 represents formula II of aryl alcohols.

FIG. 3 represents formula III of aryl substituted olefins.

FIG. 4 represents formula IV of 2-aryl propionic acid.

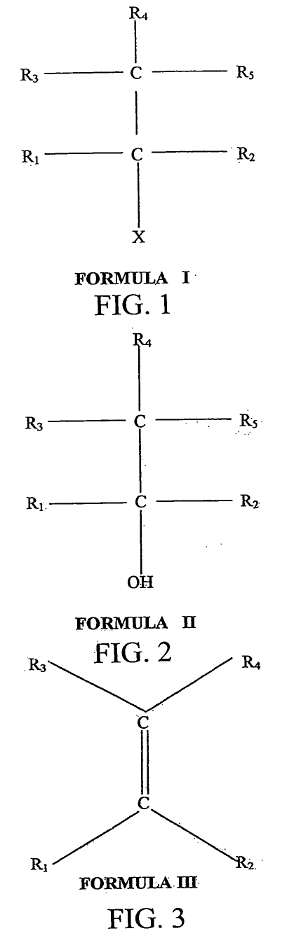

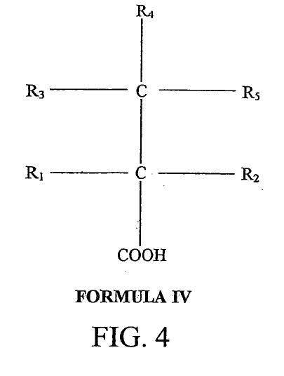

### DETAILED DESCRIPTION

Accordingly, the present invention provides an improved process for the preparation of 2-aryl propionic acids which comprises the steps of (i) reacting an aryl compound selected from arylalkyl halide having general formula I, aryl alcohol having general formula II or aryl substituted olefins having general formula III wherein, R₁ is aryl, substituted aryl, naphthyl or substituted naphthyl groups, R₂, R₃, R₄ and R₅ may independently be hydrogen, alkyl, aryl, arylalkyl, cycloaliphatic with or without substituents and X is a halogen atom selected from chlorine, bromine, iodine, with a halide promoter, and organic acid, water and a palladium catalyst, in an organic solvent selected from ketones or cyclic ethers in carbon monoxide atmosphere under homogeneous conditions, at a temperature ranging between 30 to 130° C., for a period ranging between 0.3 to 2 hrs, at pressure ranging between 50 to 1500 psig, (ii) cooling the reaction mixture to ambient temperature, (iii) flushing the reaction vessel with inert gas, (iv) removing the solvent by conventional methods and (v) separating the catalyst and isolating the compound of formula IV wherein R₁ is aryl, substituted aryl, naphthyl or substituted naphthyl groups, R₂, R₃, R₄ and R₅ are independently hydrogen, alkyl, arylalkyl, cycloaliphatic groups with or without substituents.

In one of the embodiments of the present invention, the catalyst used may be any of the palladium (0) or palladium (II) compounds, selected from palladium chloride, palladium bromide, palladium iodide, bis (triphenylphosphino) dichloro palladium (II), bis (triphenylphosphino) dibromo palladium (II), bis (triparatolylphosphino) dichloro palladium (II), bis (tricyclohexylphosphino) dichloro palladium (II), bis(triethylphosphino) dichloro palladium (II), bis (triisopropylphosphino) dichloro palladium (II), tetrakis(triphenylphosphino) palladium(0), dibenzylidieneacetonato-palladium(0), cyclooctadiene dichloro palladium(II), bisbenzonitriledichloro palladium(II), acetylacetonato palladium(II) and bisacetonitrile dichloro palladium(II).

In another embodiment, the halide promoter is selected from the group of halide salts of alkali metals comprising lithium chloride, sodium chloride, potassium chloride, lithium iodide, lithium bromide, sodium bromide, sodium iodide, potassium bromide, and potassium iodide or the group of quaternary ammonium or phosphonium halides selected from tetrabutyl ammonium chloride, tetrabutyl ammonium bromide, tetrabutyl ammonium iodide, tetrabutyl phosphonium chloride, tetrabutyl phosphonium bromide or tetrabutyl phosphonium iodide.

In still another embodiment, the organic acid is selected from the group of organic sulphonic acids such as para toluene sulphonic acid, methane sulphonic acid or triflouromethane sulphonic acid.

In yet another embodiment, the organic solvent is selected from ketones like acetone, methyl ethyl ketone, methyl isobutyl ketone, diethyl ketone, methyl n-propyl ketone, acetophenone or cyclic ethers such as tetrahydrofuran and dioxan.

In another embodiment, the concentration of the catalyst is one mole of catalyst for every 50 to 50000 moles of the compound having formula I preferably 1 mole of catalyst for every 100 to 6000 moles of the compound having formula I and more preferably one mole of catalyst for every 150 to 2000 moles of compounds having formula I, formula II or formula III.

In still another embodiment, the amount of alkali metal halide per gram mole of the catalyst is in the range of 5 to 500 moles, preferably 10 to 300 moles, and more preferably 25 to 150 moles.

In another embodiment, the amount of organic acid per gram mole of catalyst is in the range of 5 to 500 moles, preferably 10 to 300 moles, and more preferably 25 to 150 moles.

In yet another embodiment, the amount of water is in the range of 1 to 6% (v/v) of the total reaction mixture, preferably 3 to 5% (v/v).

In a feature of the invention, the reaction can be conveniently carried out in a stirred reactor with the improved catalyst system employed with a suitable solvent in presence of carbon monoxide.

In another feature of the invention, the reaction can be carried out even at low pressures of carbon monoxide (upto 50 psig).

In yet another feature of the invention, considerable enhancement in reaction rate and high selectivity towards 2-aryl propionic acids are obtained even under comparatively mild conditions.

The improved process of the present invention is described herein below with examples which are illustrative only and should not be construed to limit the scope of the present invention in any manner.

I. Conversion of Aryl Alkyl Halides to Aryl Propionic Acid

### EXAMPLE 1

A 50 ml stirred autoclave was charged with the following reactants
1-(4′-isobutylphenyl)ethyl chloride: 0.02808 mol
PdCl₂(PPh₃)₂: 5.6×10⁻⁵ mol
p-toluene sulphonic acid: 0.0056 mol
LiCl: 0.0056 mol
H₂O: 1.25 mL.
Methyl ethyl ketone: 19 mL

The contents of the autoclave were flushed with nitrogen and then many times with carbon monoxide. Thereafter, the contents were heated to 115° C. After the temperature is attained, the autoclave was pressurized to 800 psig with carbon monoxide, stirring was commenced and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the liquid phase analyzed by gas chromatography.

The GC analysis showed a turn over frequency (TOF) of 1120 h⁻¹ and 99% conversion of 1-(4′-isobutylphenyl) ethyl chloride with an ibuprofen selectivity of 95.2% and n/iso ratio of 0.05. The solvent was evaporated and the reaction mixture was re-dissolved in toluene. The solid portion, which is a mixture of LiCl and lithium salt of p-toluene sulphonic acid was filtered out. The filtrate was treated with saturated solution of sodium biocarbonate two to three times and the aqueous phase was separated which on hydrolysis with acid and extraction with dichloromethane, evaporation of solvent and vacuum distillation gives pure ibuprofen product.

### EXAMPLE 2

A 50 ml stirred autoclave was charged with the following reactants
1-(4′-isobutylphenyl)ethyl chloride: 0.056179 mol
PdCl₂(PPh₃)₂: 5.6×10⁻⁵ mol
p-toluene sulphonic acid: 0.0056 mol
LiCl: 0.0056 mol
H₂O: 1.5 mL
Methyl ethyl ketone: 15 ml

The contents of the autoclave were flushed with nitrogen and then many times with carbon monoxide. Thereafter, the contents were heated to 115° C. After the temperature is attained, the autoclave was pressurised to 800 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analysed by gas chromatography.

The GC analysis showed TOF of 1350 h⁻¹ and 99% conversion of 1-(4′-isobutylphenyl) ethyl chloride with ibuprofen selectivity 97% and n/iso ratio of 0.021. The solvent was evaporated and the reaction mixture was redissolved in toluene. The solid portion, which is a mixture of LiCl and lithium salt of p-toluene sulphonic acid was filtered out. The filtrate was treated with saturated solution of sodium bicarbonate two to three times and the aqueous phase was separated which on hydrolysis with acid and extraction with ethyl acetate, evaporation and vacuum distillation gives the pure ibuprofen product.

### EXAMPLE 3

A 50 ml stirred autoclave was charged with the following reactants
1-(4-isobutylphenyl)ethyl bromide: 0.02808 mol
PdBr₂(PPh₃)₂: 5.6×10⁻⁵ mol
p-toluene sulphonic acid: 0.0056 mol
LiBr: 0.0056 mol
H₂O: 1.25 mL
Methyl ethyl ketone: 19 ml

The contents of the autoclave were flushed with nitrogen and then many times with carbon monoxide. Thereafter, the contents were heated to 115° C. After the temperature is attained, the autoclave was pressurised to 800 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analysed by gas chromotography.

The GC analysis showed TOF of 940 h⁻¹ and 99% of 1-(4′-isobutylphenyl)ethyl bromide with ibuprofen selectivity of 95% and n/iso ratio of 0.052. The solvent was evaporated and the reaction mixture was redissolved in toluene. The solid portion, which is a mixture of LiBr and lithium salt of p-toluene sulphonic acid was filtered out. The filtrate was treated with saturated solution of sodium bicarbonate two to three times and the aqueous phase was separated which on hydrolysis with acid and extraction with dichloromethane, evaporation and vacuum distillation gives pure ibuprofen product.

### EXAMPLE 4

A 50 ml stirred autoclave was charged with the following reactants
1-(4′-isobutylphenyl)ethyl chloride: 0.02808 mol
PdCl₂(P(p-tolyl)₃)₂: 5.6×10⁻⁵ mol
p-toluene sulphonic acid: 0.0056 mol
LiCl: 0.0056 mol
H₂O: 1.25 mL
Methyl ethyl ketone: 19 ml

The contents of the autoclave were flushed with nitrogen and then many times with carbon monoxide. Thereafter, the contents were heated to 115° C. After the temperature is attained, the autoclave was pressurised to 800 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analysed by gas chromatography.

The GC analysis showed TOF of 1425 h⁻¹ and 99% conversion of 1-(4′-isobutylphenyl)ethyl chloride with ibuprofen selectivity of 95% and n/iso ratio of 0.052. The solvent was evaporated and the reaction mixture was redissolved in toluene. The solid portion, which is a mixture of LiCl and lithium salt of p-toluene sulphonic acid was filtered out. The filtrate was treated with saturated solution of sodium bicarbonate two to three times and the aqueous phase was separated which on hydrolysis with acid and extraction with dichloromethane, evaporation and vacuum distillation gave pure ibuprofen product.

### EXAMPLE 5

A 50 ml stirred autoclave was charged with the following reactants
sec Phenethyl chloride: 0.05618 mol
PdCl₂(PPh₃)₂: 5.6×10⁻⁵ mol
p-toluene sulphonic acid: 0.0056 mol
LiCl: 0.0056 mol
H₂O: 1.2 mL
Methyl ethyl ketone: 16 mL

The contents of the autoclave were flushed with nitrogen and then many times with carbon monoxide. Thereafter, the contents were heated to 115° C. After the temperature is attained, the autoclave was pressurised to 800 psig with carbon monoxide, stirring started and it was observed that gas absorption commenced immediately. For synthesis of 2-phenyl propionic acid, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 1450 h⁻¹ and 99% conversion of sec-phenethyl chloride with 2-phenyl propionic acid selectivity of 96.5% and n/iso ratio of 0.048. The solvent was evaporated and the reaction mixture was redissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane, evaporation and vacuum distallation gave pure 2-phenyl propionic acid product.

### EXAMPLE 6

A 50 ml stirred autoclave was charged with the following reactants
1-(6′-methoxy-2-naphthyl) ethyl chloride: 0.02808 mol
PdCl₂(PPh₃)₂: 5.6×10⁻⁵ mol
p-toluene sulphonic acid: 0.0056
LiCl: 0.0056 mol
H₂O: 1.25 mL
Methyl ethyl ketone: 19 mL

The contents of the autoclave were flushed with nitrogen and then many times with carbon monoxide. Thereafter, the contents were heated to 115° C. After the temperature is attained, the autoclave was pressurized to 800 psig with carbon monoxide, stirring started and it was observed that gas absorption commenced immediately. For synthesis of naproxen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 425 h⁻¹ and 99% conversion of 1-(6′-methoxy-2-naphthyl) ethyl chloride with naproxen selectivity of 97.5% and n/iso ratio of 0.025. The solvent was evaporated and the reaction mixture was re-dissolved in toluene. The solid portion, which is a mixture of LiCl and lithium salt of p-toluene sulphonic acid was filtered out. The filtrate was treated with saturated solution of sodium bicarbonate two to three times and the aqueous phase was separated which on hydrolysis with acid and extraction with dichloromethane, evaporation gave pure naproxen product.

### EXAMPLE 7

A 50 ml stirred autoclave was charged with the following reactants
1-(4′-isobutylphenyl)ethyl chloride: 0.0288 mol PdCl₂(PPh₃)₂: 5.6×10⁻⁵ mol
p-toluene sulphonic acid: 0.0056 mol
LiCl: 0.0056 mol
H₂O: 1.25 mL
Methyl ethyl ketone: 16 mL

The contents of the autoclave were flushed with nitrogen and then many times with carbon monoxide. Thereafter, the contents were heated to 115° C. After the temperature is attained, the autoclave was pressurised to 1200 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analysed by gas chromatography.

The GC analysis showed TOF of 1600 h⁻¹ and 99% conversion of 1-(4′-isobutylphenyl) ethyl chloride with ibuprofen selectivity of 99% and n/iso ratio of 0.01. The solvent was evaporated and the reaction mixture was re-dissolved in toluene. The solid portion, which is a mixture of LiCl and lithium salt of p-toluene sulphonic acid was filtered out. The filtrate was treated with saturated solution of sodium bicarbonate two to three times and the aqueous phase was separated which on hydrolysis with acid and extraction with dichloromethane; evaporation and vacuum distillation gave the pure ibuprofen product.

### EXAMPLE 8

A 50 ml stirred autoclave was charged with the following reactants
1-(4′-isobutylphenyl)ethyl chloride: 0.0288 mol
PdCl₂(PPh₃)₂: 5.6×10⁻⁵ mol
p-toluene sulphonic acid: 0.0056 mol
LiCl: 0.0056 mol
H₂O: 1.25 mL
Methyl ethyl ketone: 16 mL

The contents of the autoclave were flushed with nitrogen and then many times with carbon monoxide. Thereafter, the contents were heated to 115° C. After the temperature is attained, the autoclave was pressurized to 200 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 220 h⁻¹ and 99% conversion of 1-(4′-isobutylphenyl) ethyl chloride with an ibuprofen selectivity of 90% and n/iso ratio of 0.11. The solvent was evaporated and the reaction mixture was re-dissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane, and column chromatography gave pure ibuprofen product.

### EXAMPLE 9

A 50 ml stirred autoclave was charged with the following reactants
1-(4′-isobutylphenyl) ethyl chloride: 0.0288 mol
PdCl₂(PPh₃)₂: 5.6×10⁻⁵ mol
p-toluene sulphonic acid: 0.0112 mol
LiCl: 0.0112 mol
H₂O: 1.25 mL
Methyl ethyl ketone: 19 mL

The contents of the autoclave were flushed with nitrogen and then many times with carbon monoxide. Thereafter, the contents were heated to 115° C. After the temperature is attained, the autoclave was pressurized to 1000 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and final reaction mixture analyzed by gas chromatography.

The GC analysis showed a turn over frequency (TOF) of 1450 h⁻¹ and 99% conversion of 1-(4′-isobutylphenyl) ethyl chloride with an ibuprofen selectivity of 97% and n/iso ratio of 0.03. The solvent was evaporated and the reaction mixture was re-dissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane, evaporation and vacuum distillation gave pure ibuprofen product.

### EXAMPLE 10

A 50 ml stirred autoclave was charged with the following reactants
1-(4′-isobutylphenyl) ethyl chloride: 0.0288 mol
PdCl₂(PPh₃)₂: 5.6×10⁻⁵ mol
p-toluene sulphonic acid: 0.00562 mol
H₂O: 0.75 mL
Methyl ethyl ketone: 23 mL
LiCl: 0.00562 mol

The contents of the autoclave were flushed with nitrogen and then many times with carbon monoxide. Thereafter, the contents were heated to 115° C. After the temperature is attained, the autoclave was pressurized to 800 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the liquid phase analyzed by gas chromatography.

The GC analysis showed a turn over frequency (TOF) of 125 h⁻¹ and 99% conversion of 1-(4′-isobutylphenyl) ethyl chloride with an ibuprofen selectivity of 96.8% and n/iso ratio of 0.03. The solvent was evaporated and the reaction mixture was re-dissolved in toluene and the solid portion was filtered out. The filtrate was treated with saturated solution of sodium bicarbonate two to three times and the aqueous phase was separated which on hydrolysis with acid and extraction with dichloromethane, evaporation of the solvent and vacuum distillation gave pure ibuprofen product.

II. Conversion of Aryl Alcohols to Aryl Propionic Acid

### EXAMPLE 11

A 50 ml stirred autoclave was charged with the following reactants
1-(4′-isobutylphenyl)ethanol: 0.02808 mol
PdCl₂(PPh₃)₂: 5.6×10⁻⁵ mol
p-toluene sulphonic acid: 0.0056 mol
LiCl: 0.0056 mol
H₂O: 1.25 mL
Methyl ethyl ketone: 19 mL

The contents of the autoclave were flushed with nitrogen and then many times with carbon monoxide. Thereafter, the contents were heated to 115° C. After the temperature is attained, the autoclave was pressurized to 800 psig with carbon monoxide, stirring was commenced and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the liquid phase analyzed by gas chromatography.

The GC analysis showed a turn over frequency (TOF) of 810 h⁻¹ and 99% conversion of p-IBPE with ibuprofen selectivity of 95.2% and n/iso ratio of 0.05. The solvent was evaporated and the reaction mixture was re-dissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane and column separation gave the pure ibuprofen product.

### EXAMPLE 12

A 50 ml stirred autoclave was charged with the following reactants
1-(4′-isobutylphenyl)ethanol: 0.056179 mol
PdCl₂(PPh₃)₂: 5.6×10⁻⁵ mol
p-toluene sulphonic acid: 0.0056 mol
LiCl: 0.0056 mol
H₂O: 1.5 ml
Methyl ethyl ketone: 19 ml

The contents of the autoclave were flushed with nitrogen and then many times with carbon monoxide. Thereafter, the contents were heated to 115° C. After the temperature is attained, the autoclave was pressurized to 800 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 980 h⁻¹ and 99% conversion of p-IBPE with ibuprofen selectivity of 97% and n/iso ratio of 0.021. The solvent was evaporated and the reaction mixture was re-dissolved in toluene. The solid portion, which is a mixture of LiBr and lithium salt of p-toluene sulphonic acid was filtered out. The filtrate was treated with saturated solution of sodium bicarbonate two to three times and the aqueous phase was separated which on hydrolysis with acid and extraction with dichloromethane and column separation gave pure ibuprofen product.

### EXAMPLE 13

A 50 ml stirred autoclave was charged with the following reactants
1-(4′-isobutylphenyl)ethanol: 0.02808 mol
PdBr₂(PPh₃)₂: 5.6×10⁻⁵ mol
p-toluene sulphonic acid: 0.0056 mol
LiBr: 0.0056 mol
H₂O: 1.25 mL
Methyl ethyl ketone: 19 ml

The contents of the autoclave were flushed with nitrogen and then many times with carbon monoxide. Thereafter, the contents were heated to 115° C. After the temperature is attained, the autoclave was pressurized to 800 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 340 h⁻¹ and 99% conversion of p-IBPE with ibuprofen selectivity of 95% and n/iso ratio of 0.052. The solvent was evaporated and the reaction mixture was re-dissolved in toluene. The solid portion, which is a mixture of LiBr and lithium salt of p-toluene sulphonic acid was filtered out. The filtrate was treated with saturated solution of sodium bicarbonate two to three times and the aqueous phase was separated which on hydrolysis with acid and extraction with dichloromethane and column separation gave pure ibuprofen product.

### EXAMPLE 14

A 50 ml stirred autoclave was charged with the following reactants
1-(4′-isobutylphenyl)ethanol: 0.02808 mol
PdCl₂(P(p-tolyl)₃)₂: 5.6×10⁻⁵ mol
P-toluene sulphonic acid: 0.0056 mol
LiCl: 0.0056 mol
H₂O: 1.25 mL
Methyl ethyl ketone: 19 mL

The contents of the autoclave were flushed with nitrogen and then many times with carbon monoxide. Thereafter, the contents were heated to 115° C. After the temperature is attained, the autoclave was pressurized to 800 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 990 h⁻¹ and 99% conversion of p-IBPE with ibuprofen selectivity of 95% and n/iso ratio of 0.052. The solvent was evaporated and the reaction mixture was re-dissolved in toluene. The solid portion, which is a mixture of LiCl and lithium salt of p-toluene sulphonic acid was filtered out. The filtrate was treated with saturated solution of sodium bicarbonate two to three times and the aqueous phase was separated which on hydrolysis with acid and extraction with dichloromethane and column separation gave pure ibuprofen product.

### EXAMPLE 15

A 50 ml stirred autoclave was charged with the following reactants
Sec-Phenethyl alcohol: 0.05618 mol
PdCl₂(PPh₃)₂: 5.6×10⁻⁵ mol
p-toluene sulphonic acid: 0.0056 mol
LiCl: 0.0056 mol
H₂O: 1.2 mL
Methyl ethyl ketone: 16 ml

The contents of the autoclave were flushed with nitrogen and then many times with carbon monoxide. Thereafter, the contents were heated to 115° C. After the temperature is attained, the autoclave was pressurized to 800 psig with carbon monoxide, stirring started and it was observed that gas absorption commenced immediately. For synthesis of 2-phenyl propionic acid, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 530 h⁻¹ and 99% conversion of sec-phenethyl alcohol with 2-phenyl propionic acid selectivity of 96.5% and n/iso ratio of 0.048. The solvent was evaporated and the reaction mixture was re-dissolved in toluene. The solid portion, which is a mixture of LiCl and lithium salt of p-toluene sulphonic acid was filtered out. The filtrate was treated with saturated solution of sodium bicarbonate two to three times and the aqueous phase was separated which on hydrolysis with acid and extraction with dichloromethane and column separation gave pure 2-phenyl propionic acid product.

### EXAMPLE 16

A 50 ml stirred autoclave was charged with the following reactants
1-(6′-methoxynaphthyl)ethanol: 0.02808 mol
PdCl₂(PPh₃)₂: 5.6×10⁻⁵ mol
p-toluene sulphonic acid: 0.0056
LiCl: 0.0056 mol
H₂O: 1.25 mL
Methyl ethyl ketone: 19 mL

The contents of the autoclave were flushed with nitrogen and then many times with carbon monoxide. Thereafter, the contents were heated to 115° C. After the temperature is attained, the autoclave was pressurized to 800 psig with carbon monoxide, stirring started and it was observed that gas absorption commenced immediately. For synthesis of naproxen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 425 h⁻¹ and 99% conversion of 1-(6′-methoxynaphthyl) ethanol with naproxen selectivity of 97.5% and n/iso ratio of 0.025. The solvent was evaporated and the reaction mixture was re-dissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane and column separation gave pure naproxen product.

### EXAMPLE 17

A 50 ml stirred autoclave was charged with the following reactants
1-(4′-isobutylphenyl) ethanol: 0.0288 mol
PdCl₂(PPh₃)₂: 5.6×10⁻⁵ mol
p-toluene sulphonic acid: 0.0056 mol
LiCl: 0.0056 mol
H₂O: 1.25 mL
Methyl ethyl ketone: 19 mL

The contents of the autoclave were flushed with nitrogen and then many times with carbon monoxide. Thereafter, the contents were heated to 115° C. After the temperature is attained, the autoclave was pressurized to 1200 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 1200 h⁻¹ and 99% conversion of p-IBPE with ibuprofen selectivity of 99% and n/iso ratio of 0.01. The solvent was evaporated and the reaction mixture was re-dissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane and column separation gave pure ibuprofen product.

### EXAMPLE 18

A 50 ml stirred autoclave was charged with the following reactants
1-(4′-isobutylphenyl)ethanol: 0.0288 mol
PdCl₂(PPh₃)₂: 5.6×10⁻⁵ mol
p-toluene sulphonic acid: 0.0056 mol
LiCl: 0.0056 mol
H₂O: 1.25 mL
Methyl ethyl ketone: 16 mL

The contents of the autoclave were flushed with nitrogen and then many times with carbon monoxide. Thereafter, the contents were heated to 115° C. After the temperature is attained, the autoclave was pressurized to 200 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 190 h⁻¹ and 99% conversion of p-IBPE with ibuprofen selectivity of 90% and n/iso ratio of 0.11. The solvent was evaporated and the reaction mixture was re-dissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane and column chromatography gave pure ibuprofen product.

### EXAMPLE 19

A 50 ml stirred autoclave was charged with the following reactants
1-(4′-isobutylphenyl) ethanol: 0.0288 mol
PdCl₂(PPh₃)₂: 5.6×10⁻⁵ mol
p-toluene sulphonic acid: 0.0112 mol
LiCl: 0.0112 mol
H₂O: 1.25 mL
Methyl ethyl ketone: 19 mL

The contents of the autoclave were flushed with nitrogen and then many times with carbon monoxide. Thereafter, the contents were heated to 115° C. After the temperature is attained, the autoclave was pressurized to 1000 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 1350 h⁻¹ and 99% conversion of p-IBPE with ibuprofen selectivity of 97% and n/iso ratio of 0.03. The solvent was evaporated and the reaction mixture was re-dissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane and column separation gave pure ibuprofen product.

III. Conversion of Aryl Substituted Olefins to Aryl Propionic Acid

### EXAMPLE 20

A 50 ml stirred autoclave was charged with the following reactants
4-isobutyl styrene: 0.02808 mol
PdCl₂(PPh₃)₂: 5.6×10⁻⁵ mol
p-toluene sulphonic acid: 0.0056 mol
LiCl: 0.0056 mol
H₂O: 1.25 mL
Methyl ethyl ketone: 19 mL

The contents of the autoclave were flushed with nitrogen and then many times with carbon monoxide. Thereafter, the contents were heated to 115° C. After the temperature is attained, the autoclave was pressurized to 800 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the liquid phase analyzed by gas chromatography.

The GC analysis showed a turn over frequency (TOF) of 1512 h⁻¹ and 99% conversion of 4-isobutyl styrene with ibuprofen selectivity of 97% and n/iso ratio of 0.0293. The solvent was evaporated and the reaction mixture was re-dissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane and column separation gave pure ibuprofen product.

### EXAMPLE 21

A 50 ml stirred autoclave was charged with the following reactants
Styrene: 0.0481 mol
PdCl₂(PPh₃)₂: 5.6×10⁻⁵ mol
p-toluene sulphonic acid: 0.0056 mol
LiCl: 0.0056 mol
H₂O: 1.2 mL
Methyl ethyl ketone: 16 ml

The contents of the autoclave were flushed with nitrogen and then many times with carbon monoxide. Thereafter, the contents were heated to 115° C. After the temperature is attained, the autoclave was pressurized to 800 psig with carbon monoxide, stirring started and it was observed that gas absorption commenced immediately. For synthesis of 2-phenyl propionic acid, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 1220 h⁻¹ and 99% conversion of styrene with 2-phenyl propionic acid selectivity of 99% and n/iso ratio of 0.0101. The solvent was evaporated and the reaction mixture was re-dissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane and column separation gave pure 2-phenyl propionic acid product.

### EXAMPLE 22

A 20 ml stirred autoclave was charged with the following reactants
6-methoxynaphthyl ethene: 0.02808 mol
PdCl₂(PPh₃)₂: 5.6×10⁻⁵ mol
p-toluene sulphonic acid: 0.0056
LiCl: 0.0056 mol
H₂O: 1.25 mL
Methyl ethyl ketone: 19 mL

The contents of the autoclave were flushed with nitrogen and then many times with carbon monoxide. Thereafter, the contents were heated to 115° C. After the temperature is attained, the autoclave was pressurized to 800 psig with carbon monoxide, stirring started and it was observed that gas absorption commenced immediately. For synthesis of naproxen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 150 h⁻¹ and 99% conversion of 6-methoxynaphthyl ethene with naproxen selectivity of 98% and n/iso ratio of 0.0204. The solvent was evaporated and the reaction mixture was re-dissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane and column separation gave pure naproxen product.

### EXAMPLE 23

A 50 ml stirred autoclave was charged with the following reactants
4-isobutyl styrene: 0.02808 mol
PdBr₂(PPh₃)₂: 5.6×10⁻⁵ mol
p-toluene sulphonic acid: 0.0056 mol
LiBr: 0.0056 mol
H₂O: 1.25 mL
Methyl ethyl ketone: 19 ml

The contents of the autoclave were flushed with nitrogen and then many times with carbon monoxide. Thereafter, the contents were heated to 115° C. After the temperature is attained, the autoclave was pressurized to 800 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 540 h⁻¹ and 99% conversion of 4-isobutyl styrene with ibuprofen selectivity of 96.5% and n/iso ratio of 0.0362. The solvent was evaporated and the reaction mixture was re-dissolved in toluene. The solid portion, which is a mixture of LiBr and lithium salt of p-toluene sulphonic acid was filtered out. The filtrate is treated with saturated solution of sodium bicarbonate two to three times and the aqueous phase was separated which on hydrolysis with acid and extraction with dichloromethane and column separation gave pure ibuprofen product.

### EXAMPLE 24

A 50 ml stirred autoclave was charged with the following reactants
4-isobutyl styrene: 0.02808 mol
PdCl₂(P(p-tolyl)₃)₂: 5.6×10⁻⁵ mol
p-toluene sulphonic acid: 0.0056 mol
LiCl: 0.0056 mol
H₂O: 1.25 mL
Methyl ethyl ketone: 19 mL

The contents of the autoclave were flushed with nitrogen and then many times with carbon monoxide. Thereafter, the contents were heated to 115° C. After the temperature is attained, the autoclave was pressurized to 800 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 1680 h⁻¹ and 99% conversion of 4-isobutylstyrene with ibuprofen selectivity of 96% and n/iso ratio of 0.0417. The solvent was evaporated and the reaction mixture was re-dissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane and column separation gave pure ibuprofen product.

### EXAMPLE 25

A 50 ml stirred autoclave was charged with the following reactants
4-isobutyl styrene: 0.0288 mol
PdCl₂(PPh₃)₂: 5.6×10⁻⁵ mol
p-toluene sulphonic acid: 0.0056 mol
LiCl: 0.0056 mol
H₂O: 1.25 mL
Methyl ethyl ketone: 16 mL

The contents of the autoclave were flushed with nitrogen and then many times with carbon monoxide. Thereafter, the contents were heated to 115° C. After the temperature is attained, the autoclave was pressurized to 200 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 300 h⁻¹ and 99% conversion of 4-isobutylstyrene with ibuprofen selectivity of 92% and n/iso ratio of 0.0869. The solvent was evaporated and the reaction mixture was re-dissolved in toluene. The solid portion, which is mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane and column chromatography gave pure ibuprofen product.

### EXAMPLE 26

A 50 ml stirred autoclave was charged with the following reactants
4-isobutyl styrene: 0.0288 mol
PdCl₂(PPh₃)₂: 5.6×10⁻⁵ mol
p-toluene sulphonic acid: 0.0112 mol
LiCl: 0.0112 mol
H₂O: 1.25 mL
Methyl ethyl ketone: 19 mL

The contents of the autoclave were flushed with nitrogen and then many times with carbon monoxide. Thereafter, the contents were heated to 115° C. After the temperature is attained, the autoclave was pressurized to 1000 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 1750 h⁻¹ and 99% conversion of 4-isobutyl styrene with ibuprofen selectivity of 98.2% and n/iso ratio of 0.0183. The solvent was evaporated and the reaction mixture was re-dissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane and column separation gave pure ibuprofen product.

### EXAMPLE 27

A 250 ml stirred autoclave was charged with the following reactants
4-isobutyl styrene: 0.0288 mol
PdCl₂(PPh₃)₂: 5.6×10⁻⁵ mol
p-toluene sulphonic acid: 0.0056 mol
LiCl: 0.0056 mol
H₂O: 1.25 mL
Methyl ehtyl ketone: 19 mL

The contents of the autoclave were flushed with nitrogen and then many times with carbon monoxide. Thereafter, the contents were heated to 115° C. After the temperature is attained, the autoclave was pressurized to 1200 psig with carbon monoxide, stirring started and it was observed that gas absorption commenced immediately. For synthesis of ibuprofen, the pressure in the autoclave was maintained constant using carbon monoxide and the progress of the reaction was monitored by observing the pressure drop and by liquid sampling. The reaction was continued until the pressure drop was too low. The reactor was then cooled and the final reaction mixture analyzed by gas chromatography.

The GC analysis showed TOF of 1900 h⁻¹ and 99% conversion of 4-isobutyl styrene with ibuprofen selectivity of 99.5% and n/iso ratio of 0.005. The solvent was evaporated and the reaction mixture was re-dissolved in toluene. The solid portion, which is a mixture of LiCl and lithium salt of p-toluene sulphonic acid was filtered out. The filtrate is treated with saturated solution of sodium bicarbonate two to three times and the aqueous phase was separated which on hydrolysis with acid and extraction with dichloromethane and column separation gave pure ibuprofen product.

Advantages of the Invention:

1. Employment of a novel catalyst system under mild reaction conditions in a homogeneous medium.
2. Provides high reaction rates and high productivity of 2-aryl propionic acids (2.7 Kg/L/h)
3. Provides very high selectivity to 2-aryl propionic acids (90 to 99%) even under lower pressures of carbon monoxide (100 to 1200 psig).

1. An improved process for the preparation of 2-aryl propionic acids, the said process comprising steps of:
(i) reacting an aryl compound selected from an arylalkyl halide having formula 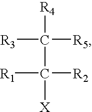
aryl alcohol having formula 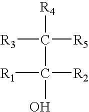
or aryl substituted olefins having formula 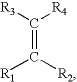
as shown in the accompanying drawings, wherein, R₁ is aryl, substituted aryl, naphthyl or substituted naphthyl groups, R₂, R₃, R₄ and R₅ are independently hydrogen, alkyl, aryl, arylalkyl or cycloaliphatic groups with or without substituents and X is a halogen atom selected from chlorine, bromine or iodine with a halide catalyst in an organic solvent selected from ketones or cyclic ethers in carbon monoxide atmosphere under homogeneous conditions, at a temperature ranging between 30 to 130° C., for a period ranging between 0.3 to 4 hrs, at pressures ranging between 50 to 1500 psig,
(ii) cooling the reaction mixture to an ambient temperature,
(iii) flushing the reaction vessel with an inert gas,
(iv) removing the solvent, and
(v) separating the catalyst and isolating 2-aryl propionic acid having a formula as follows: 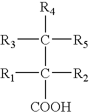
wherein, R₁ is aryl, substituted aryl, naphthyl or substituted naphthyl groups, R₂, R₃, R₄ and R₅ are independently represented by hydrogen, alkyl, aryl, arylalkyl, cycloaliphatic groups with or without substituents.

2. A process as claimed in claim 1, wherein catalyst is selected from the group of palladium(O) or palladium(II) compound comprising palladium chloride, palladium bromide, palladium iodide, bis(triparatolylphoshino) dichloro palladium(II), bis(triethylphosphino)dichloro palladium(II), bis(triisopropylphosphino)dichloro palladium(II), dibenzylidieneacetonato-palladium(O), cyclooctadiene dichloro palladium(II), bisbenzonitriledichloro palladium(II), acetylacetonato palladium(II) and bisacetonitrile dichloro palladium(II).

3. (canceled)

4. (canceled)

5. (canceled)

6. (canceled)

7. (canceled)

8. A process as claimed in claim 1 wherein the concentration of the catalyst is one mole of catalyst for every 50 to 50000 moles of the compound having the formula

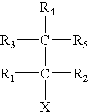

9. A process as claimed in claim 8 wherein the concentration of the catalyst is one mole of catalyst for every 100 to 6000 moles of the compound having formula

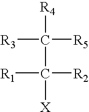

10. A process as claimed in claim 1 wherein the concentration of the catalyst is one mole of catalyst for every 150 to 2000 moles of compounds having formula 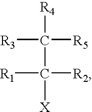
formula 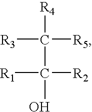
or formula

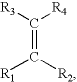

11. A process as claimed in claim 1 wherein the amount of halide promoter per gram mole of the catalyst is in the range of 5 to 500 moles.

12. A process as claimed in claim 11 wherein the amount of halide promoter per gram mole of the catalyst is in the range of 10 to 300 moles.

13. A process as claimed in claim 12 wherein the amount of halide promoter per gram mole of the catalyst is in the range of 25 to 150 moles.

14. A process as claimed in claim 1 wherein the amount of organic acid per gram mole of catalyst may be in the range of 5 to 500 moles.

15. A process as claimed in claim 14 wherein the amount of organic acid per gram mole of catalyst may be in the range of 10 to 300 moles.

16. A process as claimed in claim 15 wherein the amount of organic acid per gram mole of catalyst may be in the range of 25 to 150 moles.

17. A process as claimed in claim 1 wherein the amount of water is in the range of 1 to 6% (v/v) of the total reaction mixture.

18. A process as claimed in claim 17 wherein the amount of water is in the range of 3 to 5% (v/v) of the total reaction mixture.

19. A process as claimed in claim 1 wherein the reaction is carried out even at low pressures of carbon monoxide up to 50 psig.

20. A process as claimed in claim 7 wherein the cyclic ethers are tetrahydrofuran and dioxin.

21. A process as claimed in claim 1 wherein step (I) the organic solvent is selected from the group of ketones consisting essentially of methyl ethyl ketone, methyl isobutyl ketone, diethyl ketone, methyl n-propyl ketone, acetophenone or cyclic ethers.
